(19) United States
(12) Patent Application Publication
(10) Pub. No.: US 2012/0245387 A1
(43) Pub. Date: Sep. 27, 2012

(54) LITHIUM SALTS OF PENTAFLUOROPHENYLAMIDE ANIONS, PREPARATION THEREOF AND USE THEREOF

(75) Inventors: Jorg Sundermeyer, Marburg (DE); Thomas Linder, Cologne (DE); Bernhard Roling, Marburg (DE); Benedikt Huber, Sankt Wendel (DE); Till Fromling, Wiener Neudorf (AT)

(73) Assignee: Phillipps-UNIVERSITAT MARBURG, MARBURG (DE)

(21) Appl. No.: 13/502,894

(22) PCT Filed: Oct. 20, 2010
(86) PCT No.: PCT/EP10/65810

§ 371 Date: Jun. 19, 2012

(30) Foreign Application Priority Data
Oct. 20, 2009 (EP) 09173567.0

(51) Int. Cl. C07C 311/13; C07C 303/40

(52) U.S. Cl. 564/97

(57) ABSTRACT

The present invention provides new lithium salts comprising pentafluorophenylamide anions following the general formula Li⁺[N(SO₂—R)(C₆F₅)]⁻, which are optionally present as solvent-free complexes. R is hereby selected from fluorine, linear or branched acyclic or cyclic alkyl groups with 1 to 20 carbon atoms which are not fluorinated, partially fluorinated or fully fluorinated; or not fluorinated, partially fluorinated or fully fluorinated aryl or benzyl groups with 1 to 20 carbon atoms. The lithium salts according to the present invention are produced by reacting the corresponding NH acid of the pentafluorophenylamide with one equivalent of lithium bis(trimethylsilyl)amide or lithium organyl, wherein the reaction is carried out advantageously in the presence of apolar aprotic solvents. In this way, lithium salts in the form of solvent-free complexes are obtained. These solvent-free lithium complexes are thermally, electrochemically and against oxidation stable and comprise a high ionic conductivity. The lithium salts according to the present invention are suitable to be used as ion-conducting materials, electrically conductive materials, dyes and in chemical catalysis. They are preferably used as ion-conducting electrolytes in lithium ion accumulators.

### BACKGROUND OF THE INVENTION

1. Field of the Invention

The present invention relates to the fields of organometallic chemistry and electrochemistry.

2. Brief Description of Related Technology

With regard to rapidly progressing developments relating to accumulators based on lithium ions, there is a growing demand for lithium salts which are suitable to be used as ion-conducting electrolytes in these batteries.

The ideal candidates should be completely soluble in non-aqueous dipolar aprotic solvents or ionic liquids. If these lithium salts are dissolved in ionic liquids, they should dissociate therein into mononuclear or oligonuclear ions with high ion mobility. High transference numbers of the lithium ion are required above all. With regard to the anion, chemical and electrochemical stability are required, especially in relation to oxidative decomposition and inertness with regard to lithium in temperature ranges between 30 and 120° C.; furthermore, thermal stability and low toxicity are also required.

Until now, there is no lithium salt which fulfils all these requirements to the full extent. Among the salts used so far, LiClO₄, LiBF₄ and LiPF₆ are the most frequently used ones. Furthermore, LiN(SO₂CF₃) is considered to be highly promising.

Moreover, persons skilled in the art know lithium bis(pentafluorophenyl)amide (Li-BPFPA). Bis(pentafluorophenyl)amine (BPFPA) is suitable to be obtained, for example, by reacting LiNH₂ with C₆F₆. This is described in R. Koppang: “Use of a Lithium Amide Suspension in Tetrahydrofuran for Preparation of Some Polyfluorophenyl- and Polyfluorodiphenylamines”, Acta Chem Scand 1971, 25, 3067-3071. The lithium salt of BPFPA is suitable to be obtained by reacting BPFPA with n-butyllithium in hexane. Li-BPFPA dissolves only very little in hydrocarbons, but is highly soluble in dipolar aprotic solvents or ether. This is described in A Khvorost, P L Shutov, K Harms, J Lorberth, J Sundermeyer, S S Karlov, G S Zaitseva: “Lithium Bis(pentafluorophenyl)amides—Synthesis and Characterization of its Complexes with Diethyl Ether and THF”, Z. Anorg. Allg. Chem. 2004, 630, 885-889. Etherates are, however, not suitable for thermal applications and electrochemical applications in particular.

WO 2009/003224 A1 describes lithium energy storage devices which comprise an ionic liquid electrolyte comprising bis(fluorsulfonyl)imide as the anion, a cation which functions as a counterion, and lithium ions. Lithium salts comprising pentafluorophenylamide anions are not disclosed.

U.S. Pat. No. 6,319,428 B1 describes ion-conducting substances with anions following the general formula [R_{f}—SOₓ—N—Z]⁻. Rf is hereby a perfluorinated group, but never a pentafluorophenyl group; Z is an electron withdrawing group, SOₓ stands for a sulfonyl or sulfinyl group, and monovalent metal cations are used as counterions. Lithium salts of the anions described are prepared via ion exchange from the corresponding potassium salt by adding lithium chloride to THF.

US 2007/0093678 A1 describes perfluoroalkylsulfonamide compounds of the general formula Y⁺[N(SO₂R^{f})(CF₃)]⁻. Therein, Y⁺ is an arbitrary organic or inorganic cation, and R^{f} is a perfluorinated alkyl group with 1 to 4 carbon atoms. Under no circumstances does the anion comprise perfluorinated aryl groups.

In T Linder, J Sundermeyer: “Three novel anions based on pentafluorophenyl amine combined with two new synthetic strategies for the synthesis of highly lipophilic ionic liquids”, Chem Commun 2009, 2914-2916, in which are described ionic liquids with bis(pentafluorophenyl)amide, pentafluorophenyl(trifluoromethylsulfonyl)imide and pentafluorophenyl(nonafluorobutylsulfonyl)imide which function as anions, and imidazolium and phosphonium ions which function as cations. The article, however, does not provide any indication as to the preparation of ether-free lithium pentafluorophenylimide compounds or to their thermal and electrochemical properties.

WO 95/26056 describes ion-conducting materials which comprise at least one ionic compound in an aprotic solvent, wherein the ionic compound is a compound of the formula (1/mM)⁺[(ZY₂)N]⁻. Therein, M is a metal, m is its valence, Y is SO₂ or POZ, and each substituent Z is a fluorine atom independent of one another, an optionally perfluorinated organic group which optionally comprises at least one polymerizable group, wherein at least one of the substituents Z represents a fluorine atom. This patent application does not, however, disclose any compounds in which a pentafluorophenyl group is directly bound to a nitrogen atom.

WO 2007/131498 A2 describes hydrophobic ionic liquids from pentafluorophenylimide ions and organic or inorganic cations, wherein inorganic cations are selected from alkali or alkaline earth metal cations. However, there is no indication as to the particular suitability of lithium pentafluorophenylamide ions to be used as ion-conducting electrolytes in lithium ion accumulators, and the production methods described therein use, inter alia, ether.

### SUMMARY OF THE INVENTION

The present invention overcomes the disadvantages of the state of the art by providing a new class of lithium salts with weakly coordinating anions. These new lithium salts are formally derived from the lithium salts of the di(perfluoroalkylsulfonyl)amides and di(fluoroalkylsulfonyl)amides by replacing a perfluoroalkylsulfonyl group or a fluorosulfonyl group with a pentafluorophenyl group (Pfp) which withdraws electrons. Furthermore, the present invention provides a new method to prepare these lithium salts whilst being free from coordinating ether molecules. The new lithium salts according to the present invention are thermally more stable and comprise higher ion mobility than known lithium salts.

### BRIEF DESCRIPTION OF THE DRAWING FIGURES

FIGS. 1-3 are ORTEP plots of compounds according to the invention.

FIG. 4 illustrates a thermogravimetric analysis of compounds according to the invention.

FIG. 5 illustrates the conductivity spectra of a compound according to the invention.

FIG. 6 is an Arrhenius plot of the conductivity of compounds according to the invention.

FIG. 7 is a Meyer-Neldel plot of pre-exponential factors and activation energies of compounds according to the invention.

FIGS. 8-10 are cyclic voltammograms of compounds according to the invention.

### DETAILED DESCRIPTION OF THE INVENTION

The present invention provides new pentafluorophenylamide anions comprising lithium salts and a method for their production. According to the present invention, these lithium salts are prepared by reacting the corresponding NH acid of the pentafluorophenylamide with one equimolar amount of lithium bis(trimethylsilyl)amide or lithium organyl. The reaction is carried out in the presence of non-polar aprotic or dipolar aprotic solvents. If non-polar aprotic solvents are used, lithium salts are obtained which exist as solvent-free complexes. Due to the absence of solvents such as ether, the lithium salts according to the present invention comprising pentafluorophenylamide anions are thermally and with regard to oxidation more stable than the respective solvent-containing complexes, and are suitable to be used as ion-conducting materials, electrically conductive materials, dyes and in chemical catalysis. They are preferably used as ion-conducting electrolytes in lithium ion accumulators.

The aim of the present invention is to provide new lithium salts with improved thermal stability and improved ion mobility, as well as methods for their preparation.

The aim of providing new lithium salts with improved thermal stability and improved ion mobility is achieved according to the present invention by lithium salts that include a lithium cation and a pentafluorophenylamide anion according to formula (I)

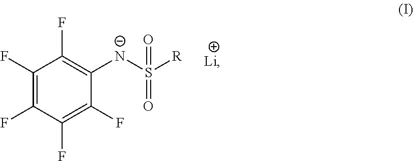

wherein R is selected from
F (fluoride),
linear or branched acyclic or cyclic alkyl groups with 1 to 20 carbon atoms which are not fluorinated, partially fluorinated or completely fluorinated.
not fluorinated, partially fluorinated or fully fluorinated aryl or benzyl groups with up to 20 carbon atoms.

Surprisingly, it has been found that the lithium salts comprising pentafluorophenylamide anions comprise higher thermal stability and higher ion mobility than lithium salts known so far. The compounds according to the present invention and formula (I) are hereinafter also referred to as lithium salts of pentafluorophenylamide.

The lithium salts of pentafluorophenylamide according to the present invention and the method for their preparation are explained hereinafter, wherein the invention comprises all of the embodiments presented hereinafter individually and in combination with one another.

In a preferred embodiment, R is a fluorine atom.

In another preferred practical embodiment, R is a non-fluorinated, partially fluorinated or fully fluorinated alkyl group with 1 to 20 carbon atoms, wherein the alkyl group is suitable to be linear or branched, cyclic or acyclic. Particularly preferably, it refers to a fully fluorinated, i.e. a perfluorinated alkyl group.

Within the context of the present invention, linear and branched acyclic alkyl groups with 1 to 20 carbon atoms are selected from methyl, ethyl, n-propyl, isopropyl, 1-butyl, 2-butyl, tert-butyl, 1-pentyl, 2-pentyl, 3-pentyl, 3-methylbutyl, 2,2-dimethylpropyl, and all the isomers of hexyl, heptyl, octyl, nonyl, decyl, undecyl and dodecyl, tridecyl, tetradecyl, pentadecyl, hexadecyl, heptadecyl, octadecyl, nonadecyl, eicosyl.

It is known to persons skilled in the art that cyclic alkyl groups have to comprise at least three carbon atoms. Within the context of the present invention, cyclic alkyl groups therefore comprise propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl rings with 3 to 20 carbon atoms. In the context of the present invention, a cyclic alkyl group is selected from the annular alkyl groups mentioned which do not carry any further substituents, and from the annular alkyl groups which, for their part, are bound to one or several acyclic alkyl groups. In the case of the latter, the binding of the cyclic alkyl group to the SO2 group in accordance with formula I is suitable to occur via a cyclic or an acyclic carbon atom of the cyclic alkyl group. According to the definition above of the term “alkyl group”, cyclic alkyl groups also comprise a total of 20 carbon atoms maximum.

In another preferred practical embodiment, R is a non-fluorinated, partially fluorinated or fully fluorinated aryl or benzyl group with up to 20 carbon atoms. Aryl groups are hereby selected from phenyl, naphthyl, anthracenyl, phenanthrenyl, tetracenyl, biphenyl, terphenyl. Particularly preferably, it refers to a fully fluorinated, i.e. a perfluorinated benzyl or aryl group.

The aryl or benzyl group is suitable to be optionally substituted with one to three linear or branched alkyl groups, wherein these alkyl groups are not suitable to be fluorinated, partially fluorinated or fully fluorinated. The aryl or benzyl groups substituted with one to three alkyl groups hereby also comprise up to 20 carbon atoms.

R is very particularly preferably selected from F, trifluoromethyl and nonafluoro-n-butyl.

In particularly advantageous embodiments of compounds in accordance with formula I, no solvent molecule is coordinated to the lithium cation; in particular no ether molecules are coordinated to the lithium cation.

The lithium salts of pentafluorophenylamide according to the present invention and formula (I) are prepared by reacting the free acid of the corresponding pentafluorophenylamide in accordance with formula (II) with equimolar amounts of lithium bis(trimethylsilyl)amide or a lithium organyl. This is shown, by way of example, for the reaction with lithium bis(trimethylsilyl)amide:

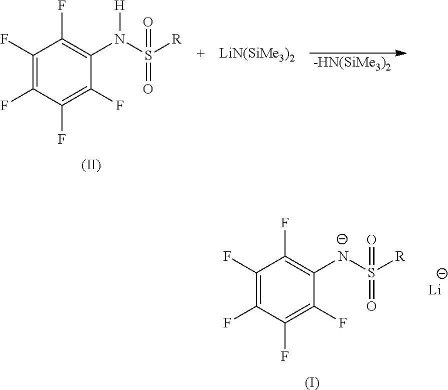

The reaction is carried out at a temperature of approximately 20 to 80° C. during 12 hours under stirring in a non-polar aprotic or dipolar aprotic solvent. The free acid of the lithium (pentafluorophenyl)amide is hereby dissolved in the solvent by heating to 30 to 60° C. Lithium bis(trimethylsilyl)amide or a lithium organyl such as tert.-butyllithium is subsequently added. It is particularly advantageous to work with lithium bis(trimethylsilyl)amide, which is dissolved beforehand in the same solvent as the free acid of the lithium (pentafluorophenyl)amide. Lithium bis(trimethylsilyl)amide or the lithium organyl is also suitable to be optionally added as a solid to the solution of the free acid of the lithium (pentafluorophenyl)amide.

All compounds which comprise a lithium carbon binding and which are not lithium cyanide are hereby to be understood under “lithium organyl”.

Both reactants are not necessarily required to be dissolved in the same solvent. The sequence of adding is also suitable to be carried out in reverse order, i.e. the lithium bis(trimethylsilyl)amide or lithium organyl is provided, and the NH acid is added. The total concentration of the two reactants in the aforementioned solvents is hereby advantageously 0.01 mol/L to 0.5 mol/L.

It is recommended to firstly dissolve the two reactants and subsequently mix them with one another as described above. In this case it is advantageous to use approximately one half to two-thirds of the solvent, the total of which is to be used to dissolve the free acid of the pentafluorophenylamide, and to dissolve either the lithium bis(trimethylsilyl)amide or the lithium organyl with the residual solvent.

After 12 h at 80° C., the reaction mixture is cooled to room temperature. The volatile components are subsequently removed, and the residue is washed and dried with a non-polar aprotic or dipolar aprotic solvent. The removal of the volatile components and the drying are carried out advantageously at reduced pressure.

The non-polar aprotic or dipolar aprotic solvent is selected from aliphatic, unsaturated and aromatic hydrocarbons such as toluene, partially or fully halogenated hydrocarbons (e.g. chlorobenzene, chloroform, carbon tetrachloride, CFC, FC, chlorofluorcarbon (Frigene) and petroleum ether. Toluene is the preferred solvent. Dipolar aprotic solvents, in which the reaction is also suitable to be carried out, are, by way of example, diethyl ether, tetrahydrofuran, and other cyclic and acyclic ethers. Also suitable are DMF, DMSO, cyclic and acyclic diorganocarbonates, carboxylic acids, carboxylic acid diorganoamides, pyridine and tertiary amines. In dipolar aprotic solvents, complexes of the lithium cation are in general isolated by means of the donor functions of these solvents, for example lithium etherates. The synthesis in apolar aprotic solvents is therefore to be preferred to the synthesis in dipolar aprotic solvents.

However, it is known to persons skilled in the art that solvent ligands—for example ether ligands—can be replaced at the lithium atom by other ligands, such as ionic liquids or organocarbonates. By means of this displacement reaction, solvent-free lithium salts according to the present invention and in accordance with formula I are suitable to be produced from the corresponding complexes coordinated with solvents.

“Reduced pressure” is a pressure below the standard pressure level of 1,013 mbar. It is advantageous to remove the volatile components with the help of a vacuum of 0.000001 mbar to 10 mbar.

The non-polar aprotic oxygen-free solvents mentioned above are suitable for washing the residue. Particularly suitable are for example n-pentane, cyclopentane, n-hexane, cyclohexane and n-heptane.

The method according to the present invention therefore comprises the following steps:
a) dissolving the free acid of the pentafluorophenylamide in accordance with formula II

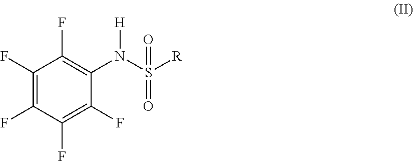

in a non-polar aprotic or dipolar aprotic solvent by heating,
b) mixing with an equimolar amount of lithium bis(trimethylsilyl)amide or a lithium organyl as a solid or in solution to obtain a mixture,
c) adjusting the obtained mixture of free acid consisting of the pentafluorophenylamide and the lithium trimethylsilylamide or the lithium organyl to a total concentration of these two reactants from 0.01 mol/L to 0.5 mol/L,
d) stirring the mixture at 80° C. for 12 hours,
e) cooling the mixture to room temperature,
f) removing the volatile components from the mixture to provide a residue,
g) washing the residue with a non-polar aprotic or dipolar aprotic solvent and subsequent drying.

In a preferred embodiment, the lithium bis(trimethylsilyl)amide or the lithium organyl in accordance with step b) is added as a solution.

In another embodiment, the lithium bis(trimethylsilyl)amide or the lithium organyl in accordance with step b) is added as a solid.

In another embodiment, the mixing in accordance with step b) occurs by providing a solution of the lithium bis(trimethylsilyl)amide or the lithium organyl and subsequently adding the solution of the pentafluorophenylamide in accordance with step a).

Very particularly preferred is if the method according to the present invention is carried out using non-polar aprotic solvents. This refers in equal measure to the dissolution of the free acid of the pentafluorophenylamide in accordance with formula II (in step a) and the optional dissolution of the lithium bis(trimethylsilyl)amide or the lithium organyl in step b) and the washing of the residue in accordance with step g).

The method according to the present invention is advantageous, as it provides the lithium salts of pentafluorophenylamide according to the present invention in high yields, and only one mol of lithium bis(trimethylsilyl)amide or lithium organyl has to be applied per mol of the desired product. LiN(SiMe₃)₂ and lithium organyls are less nucleophilic bases than the corresponding NH acid of the pentafluorophenylamide.

The production and structural, thermal and electrical properties of two lithium salts according to the present invention are described in the practical embodiments 1 to 13. These lithium salts are lithium pentafluorophenyl(trifluoromethylsulfonyl)imide (Li-PFTFSI, 3) and lithium pentafluorophenyl(nonafluorobutylsulfonyl)imide (Li-PFNFSI, 4). Production and properties of these two lithium salts according to the present invention are compared to lithium bis(pentafluorophenyl)amide (Li-BPFPA, 1), from which the 5,10-bis(pentafluorophenyl)-5,10-dihydro-octafluorophenazine (2) and lithium bis(trifluoromethylsulfonimide) (Li-TFSI, LiN(SO₂CF₃)₂) are obtainable by heating. The substances 1, 2 and Li-TFSI are known by the state of the art; Li-TFSI is a standard electrolyte known to persons skilled in the art.

The thermal stability of (1) is described in practical embodiment 11. In comparison to the standard electrolyte Li-TFSI, which decomposes at 360° C., Li-BPFPA (1) only has limited thermal stability. This is shown in practical embodiment 2. Within the context of the present invention, new lithium salts were therefore produced based on the asymmetrically substituted sulfonamides (F₅C₆)N(H)SO₂CF₃ (H-PFTFSI) and (F₅C₆)N(H)SO₂C₄F₉ (H-PFNFSI). These two compounds are strong NH acids, comparable with the corresponding acid HN(SO₂CF₃)₂, and form a series of ionic liquids or even crystalline hydroxonium salts if they are crystallized from water-saturated ether.

The lithium salts according to the present invention and the principally known substance Li-BPFPA (1) were produced within the context of the present invention, firstly by reacting lithium bis(trimethylsilyl)amide LiN(SiMe₃)₂with the respectively corresponding NH acid. This new method allows for the first time for the ether-free production of lithium salts comprising pentafluorophenylamide anions. The lithium salts according to the present invention, which for the first time are not present as etherates, are, due to their improved thermal and electrochemical properties, more suitable to be used as electrically conductive electrolytes than the corresponding etherates.

The compounds according to the present invention Li-PFTFSI 3 and Li-PFNFSI 4 were obtained in the form of colorless powders which are insoluble in toluene and other hydrocarbons. They are, however, highly soluble in dipolar aprotic solvents such as diethyl ether, THF, dimethylcarbonate, DMF and DMSO.

The results from the impedance spectroscopic measurements (see practical embodiment 13) show a correlation between the activation entropy S_{A}^{act} and the symmetry of the anions. The TFSI anion is the most symmetrical of these anions, which leads to a high packing density of the anions in the crystal. A high packing density is advantageous for the coordination of the lithium ions via oxygen and therefore for the existence of clearly defined lithium lattice sites with low potential energy. In the thermal equilibrium, the configurational entropy of the lithium ions is low on these lattice sites. Lithium ions occupy the interstitial sites via thermal activation, leading to the formation of holes. The interstitial occupation of lithium ions and the formation of holes lead to an increase in the potential energy and configurational entropy. This is reflected in high activation energy and high activation entropy for lithium ion conduction in Li-TFSI.

The replacement of the TFSI anions by the less symmetrical PFTFSI anions leads to a lower packing density for the anions and to a less favorable coordination of the lithium ions. The lithium ions in Li-PFTFSI therefore have higher potential energy and higher configurational entropy in comparison to Li-TFSI. This leads to lower activation energy and entropy values for the transport of lithium ions.

The results of the impedance spectroscopy for Li-PFNFSI also show that due to the low symmetry of the PFNFSI anions, there is no clear distinction between lithium ions on well-defined lattice sites and defects such as interstitial ions and holes. The number of available sites for lithium ion conductivity is higher than the number of lithium ions itself, thereby leading to negligible energy and entropy for the formation of movable ions.

The ionic conductivity was measured for the substances Li-TFSI, Li-PFTFSI and Li-PFNFSI. Li-PFTFSI comprises the highest conductivity of these three substances in the temperature window measured. It is apparent from FIG. 7 that the activation energy E_{A}^{act} for the formation of movable lithium ions is lower in this salt, as was to be expected due to an interpolation of the Meyer-Neldel data between PFNFSI and Li-TFSI. If TFSI anions are therefore replaced by PFTFSI anions, the activation entropy decreases due to the lower symmetry of the anions; however, the activation energy decreases to an even higher degree, as was to be expected on the basis of the interpolation. The lower symmetry of the PFTFSI anions in comparison to TFSI anions leads to an increase in the configurational entropy of the lithium ions, but the more significant effect is the decrease in the activation energy for the formation of holes. The coordination of the lithium ions in Li-PFTFSI is significantly lower than in Li-TFSI. However, the comparably high activation entropy shows that there is a clear distinction between lithium ions on regular lattice sites and mobile holes. This manner of interplay between energetic and entropic factors leads to a higher ionic conductivity of Li-PFTFSI in comparison to the other two ions.

It is readily apparent to persons skilled in the art that similar conclusions apply for other lithium salts according to the present invention with regard to symmetry, coordination of the lithium ions, ionic conductivity, increase in configurational entropy and decrease in the activation energy for the holes.

The lithium salts according to the present invention are suitable to be used as ion-conducting materials, electrically conductive materials, dyes and in chemical catalysis. They are preferably used as ion-conducting electrolytes in lithium ion accumulators. Lithium ion accumulators are also referred to as lithium ion batteries.

### Practical Embodiments

### Practical Embodiment 1

### Production of lithium bis(pentafluorophenyl)amide Li-BPFPA (1)

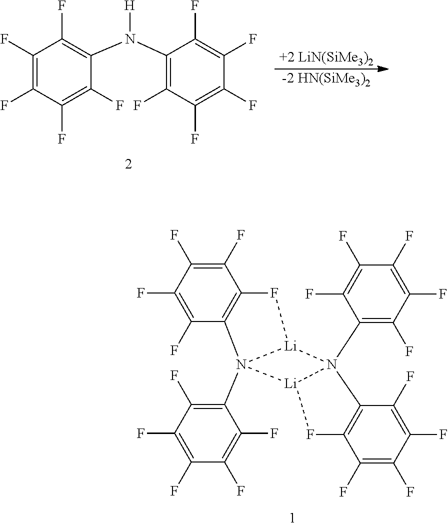

0.97 g (2.77 mmol) of bis(pentafluorophenyl)amide (BPFPA-H) was dissolved under heating in 20 ml of toluene in a Schlenk flask. A solution of 0.47 g (2.77 mmol) LiN(SiMe₃)₂ in 10 ml of toluene was subsequently added by means of a syringe. The suspension obtained in this way was stirred for 12 h at 80° C. All volatile components were removed at reduced pressure; the residue was washed with hexane and dried in vacuum. 0.54 g (55%) of 1 was obtained in the form of a colorless powder. Crystals of radiographic quality were obtained from a saturated toluene solution at −30° C.

¹⁹F-NMR ([D₆]-DMSO, 282 MHz): δ=−185.7 nm (m, 2F, p-F), −170.10 (pt, 4F, m-F), −160.6 ppm (pd, 4F, o-F)

IR (Nujol): 570(w), 722(w), 823(w), 964(w), 999(m), 1031(s), 1179(w), 1305(w) 1520 cm⁻¹(s)

ESI-MS (negative mode): m/z (%): 348.0 (100%) [BPFPA⁻]

Elementary analysis: calcd. (%) for C₁₂F₁₀LiN: C 40.56, N 3.94; found: C 40.19, N 4.04

### Practical Embodiment 2

### Production of 5,10-bis(pentafluorophenyl)-5,10-dihydro-octafluorophenazine (2) by Means of Unfavorable Thermolysis

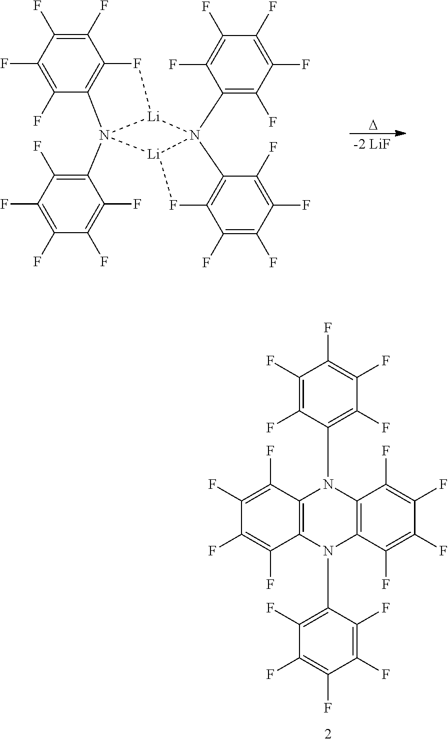

A glass tube was charged with 0.50 g of the substance 1 and placed in an electric oven. The tube was connected to the vacuum pipe (10⁻² mbar), and the oven was slowly heated to 220° C. In the cooler area of the tube, a yellowish sublimate was observed. The sublimate was collected and washed with warm hexane in order to remove the impurities caused by BPFPA-H. The colorless solid obtained in this way was dried in vacuum. 0.12 g of the compound 2 was obtained. Crystals of radiographic quality were obtained from a solution in a mixture of hexane and toluene at −30° C. (approx. 1:4).

¹⁹F-NMR ([D₆]-DMSO, 282 MHz): δ=−161.4 (pdt, 4F, m-F, C₆F₅), −160.8 (pdd, 4F, NCCFCF, phenazine), −154.9 (pdd, 4F, NCCF, phenazine), −149.6 (pt, 2F, p-F, C₆F₅), −141.6 ppm (ps, 4F, o-F, C₆F₅)

IR (KBr): 479(m), 608(m), 666(w), (684(w), 765(m), 822(m), 929(m), 995(m), 1067(s), 1097(s), 1166(w), 1190(w), 1260(w), 1321(w), 1443(m), 1509(s), 1619(s), 1637 cm⁻¹(s)

MS (70 eV): m/z(%): 658 (80%),[M⁺], 491 (100%) [M⁺-C₆F₅];

Elementary analysis: calcd. (%) for C₂₄F₁₈LiN₂: C 43.79, N 4.26; found: C 43.83, N 4.29

### Practical Embodiment 3

### Production of lithium pentafluorophenyl(trifluoromethylsulfonyl)imide Li-PFTFSI (3)

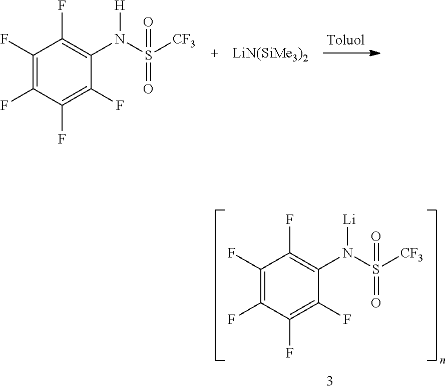

0.49 g (1.54 mmol) of pentafluoro(trifluoromethylsulfonyl)imide (PFTFSI-H) was dissolved under heating in 20 ml of toluene in a Schlenk flask. A solution of 0.25 g (1.54 mmol) LiN(SiMe₃)₂ in 10 ml of toluene was subsequently added by means of a syringe. The suspension obtained in this way was stirred for 12 h at 80° C. All volatile components were removed at reduced pressure, and the residue was washed with hexane. After drying in vacuum, the compound 3 was obtained in the form of a colorless powder. Yield: 0.44 g (88%).

¹⁹F-NMR ([D₆]-DMSO, 282 MHz): δ=−168.2 (pt, 1F, p-F), −166.8 (pt, 2F, m-F), −146.1 (d, 2F, o-F), −77.5 ppm (t, 3F, CF₃);

IR (Nujol): 556(w), 608(w), 722(w), 801(w), 909(m), 988(s), 1049(s), 1123(s), 1203(s), 1262(m), 1332(m), 1507 cm⁻¹(s)

ESI-MS (negative mode): m/z (%): 314.0 (100%) [PFTFSI⁻];

Elementary analysis: calcd. (%) for C₇F₈LiNO₂S: C 26.17, N 4.36; found: C 25.41, N 4.82

### Practical Embodiment 4

### Production of lithium pentafluorophenyl(nonafluorobutylsulfonyl)imide Li-PFNFSI (4)

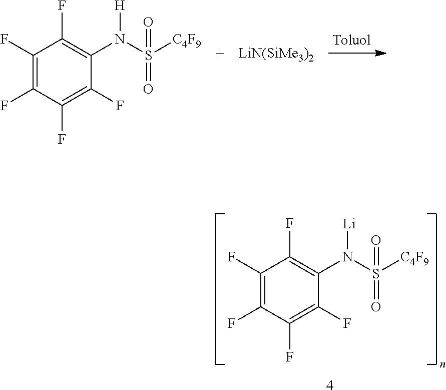

1.11 g (2.38 mmol) of pentafluoro(nonafluorobutylsulfonyl)imide (PFNFSI-H) was dissolved under heating in 20 ml of toluene in a Schlenk flask. A solution of 0.40 g (2.38 mmol) LiN(SiMe₃)₂ in 10 ml of toluene was subsequently added by means of a syringe. The suspension obtained in this way was stirred for 12 h at 80° C. All volatile components were removed at reduced pressure, and the residue was washed with hexane. After drying in vacuum, the compound 4 was obtained in the form of a colorless powder. Yield: 0.99 g (88%).

¹⁹F-NMR ([D₆]-DMSO, 282 MHz): δ=−168.2 (pt, 1F, p-F), −166.8 (pt, 2F, m-F), −149.8 (pd, 2F, o-F), −125.8 (s, 2F, SO₂CF₂), −120.8 (t, 2F, SO₂CF₂CF₂), −114.1 (t, 2F, F₃CCF₂), −80.4 ppm (t, 3F, CF₃);

IR (Nujol): 501(w), 593(m), 722(w), 784(w), 901(m), 989(s), 1056(s), 1136(s), 1060(s), 1211(s), 1310(s), 1518 cm⁻¹(s)

ESI-MS (negative mode): m/z (%): 464.0 (100%) [PFNFSI⁻]

Elementary analysis: calcd. (%) for C₁₀F₁₄LiNO₂S: C 25.48, N 2.97; found: C 24.40, N 2.63

### Practical Embodiment 5

### Production of the THF Complex of lithium pentafluorophenyl(nonafluorobutylsulfonyl)imide [Li(THF)₂(PFNFSI)]4(THF)₂

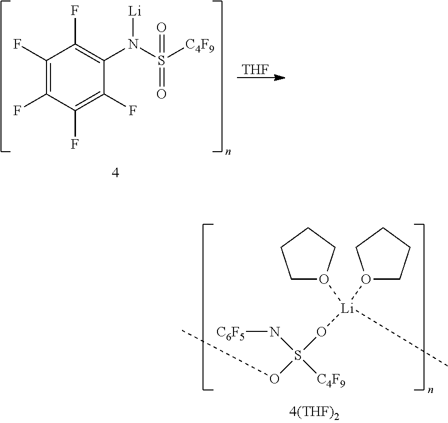

The recrystallization of compound 4 from practical embodiment 4 into THF at −30° C. results in colorless crystals of [Li(THF)₂(PFNFSI)], which are suitable for the X-ray structure analysis.

X-Ray Crystallography

The X-ray diffraction intensities were measured on a STOE IPDS-I- and IPDS-II-diffractometer system by using Mo—K_{α}-radiation (0.71073 A). The structures were determined with the help of direct methods, completed by means of a subsequent difference Fourier synthesis, and refined in accordance with the method of the least squares in the full matrix method. All non-hydrogen atoms were refined with anisotropical deflection coefficients. The hydrogen atoms were calculated and isotropically refined. The following programs were used: WinGX, SIR-97, SHELXL-97.

### Practical Embodiment 6

### X-ray crystallography of lithium bis(pentafluorophenyl)amide Li(BPFPA) (1)

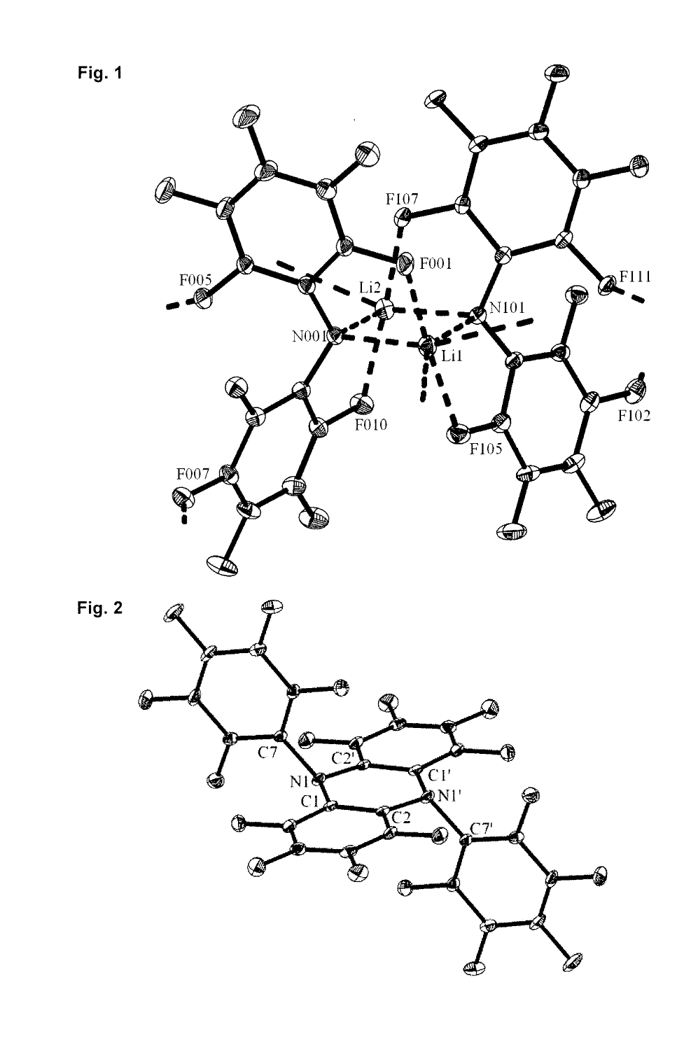

Li(BPFPA) 1 crystallizes in the space group P 2₁/c with Z=8. In solid state it forms a dimer. The two lithium atoms are bound by means of the two nitrogen atoms of the bridging amide ligands by forming an almost planar Li₂N₂-unit. Each nitrogen atom forms a shorter (2.08 Å) and a longer (2.10 Å or 2.11 Å) Li—N-bond. These bond lengths are within 3σ identical to the structures of the etherates. In contrast to the etherates, however, the lithium atoms in 1 complete their coordination spheres exclusively with a set of weak contacts to fluorine atoms. Each lithium atom is sixfold coordinated by means of two nitrogen atoms and four fluorine atoms. The four intramolecular Li—F-contacts of the dimer in FIG. 1 are short and vary in the area of 2.02 Å bis 2.22 Å. One of the two short Li—F-contacts of each lithium atom derives from the ortho-fluorine atoms of the pentafluorophenyl unit (Pfp) of an amide ligand, whereas the other is formed by the ortho-fluorine atom of the Pfp unit of the adjacent amide ligand of the dimer unit. In this regard, the ligand BPFPA is suitable to be considered as a κ³-(N,F,F)-bridging unit. The intermolecular Li—F-contacts complete the distorted octahedral coordination of the lithium and are primarily oriented in the equatorial Li₂N₂-plane. They are somewhat longer than the intramolecular distances and comprise contact distances of up to 2.60 Å or 2.67 Å. As a consequence of the intermolecular Li—F-contacts, a three-dimensional network is formed in which each dimer unit is coordinated via eight coordinative bonds with four adjacent units.

### Practical Embodiment 7

### X-ray Crystallography of 5,10-bis(pentafluorophenyl)-5,10-dihydro-octafluorophenazine (2)

5,10-bis(pentafluorophenyl)-5,10-dihydro-octafluorophenazine 2 crystallizes in the space group P1;⁻ with Z=1. The molecule comprises a crystallographically caused inversion symmetry. The molecular structure of 2 comprises a planar arrangement of the atoms of the central dihydrophenazine unit. It was found that the angle between both planes, which is defined by the two aryl rings, is an ideal angle of 180° C. This is in contrast to the non-fluorinated derivatives, in which the dihydrophenazine unit is folded in the N—N axis, as for example in 5,10-dimethyl-5,10-dihydrophenazine, which comprises an interplanar angle of 144°, and in 2,7-dibromo-1,6-dichloro-5,10-dimethyl-5,10-dihydrophenazine, whose interplanar angle is 152°. Due to steric reasons and as a consequence of the planarity of the central ring, the Pfp substituents at the nitrogen atom deviate from the plane and take over an anti-arrangement in relation to each other. The angle between the C7-N1-bond and the plane of the dihydrophenazine was determined to be 41°. The bond lengths of the C—N bonds in 2 were determined to be 1.41 Å and 1.43 Å and lie in the same range as in the non-fluorinated dihydrophenazines, for which values of 1.41 Å for 5,10-dimethyl-5,10-dihydrophenazine and values between 1.37 Å and 1.39 Å for 2,7-dibromo-1,6-dichloro-5,10-dimethyl-5,10-dihydrophenazine are reported in the literature.

In summary, the properties of the perfluorinated dihydrophenazine comprise a large structural difference to the non-fluorinated, electron-rich parent compounds.

### Practical Embodiment 8

### X-ray Crystallography of the Compounds (3) and (4)

As the compounds 3 and 4 are virtually insoluble in non-coordinating solvents, they were re-crystallized using THF.

In the case of 4, crystals of radiographic quality were obtained from a THF solution at −30° C. The structure analysis discloses a structure which comprises two THF molecules per Li-PFNFSI; see practical embodiment 5. 4(THF)₂ crystallizes in the space group P1;⁻ with Z=1, and forms a chain structure in which the lithium atoms are exclusively coordinated by means of oxygen atoms; no Li—N or Li—F contacts were able to be observed. All lithium cations are tetrahedrally coordinated by means of two oxygen atoms of the sulfonyl groups of the PFNFSI anions and two oxygen atoms of the THF molecules. For the distances between the lithium and oxygen atoms, values between 1.92 and 1.96 Å were determined. These distances are marginally shorter than in the structurally-related compound [LiN(SO₂CF₃)₂(DME)]. DME hereby stands for dimethoxyethane. As no particularly short Li—O distances were determined, there are only weak interactions between cation and anion. The nitrogen atom, which formally carries the negative charge, does not show any interaction with the cation. In actual fact, delocalization of the negative charge primarily takes place via the sulfonyl unit. The determined comparably short S—N distance of 1.52 Å, which is in the area of an S—N double bond, is in accordance with this delocalization. In comparison to the reference compound [LiN(SO₂CF₃)₂(DME)] (1.57 Å), the S—N bond length in 4(THF)₂ is shorter, because the negative charge on the nitrogen atom in the case of 4(THF)₂ is delocalized to a greater extent in the THF group than in the pentafluorophenyl group. Furthermore, with a value of 121°, the bond angle on the nitrogen atom indicates an S—N double bond character.

### Practical Embodiment 9

### Hydrolysis of the Compound (1)

The stability of 1 against hydrolysis was examined. A solution of 1 in [D₆]-DMSO yields a signal at δ_{F}=−185.7 ppm for the para-fluorine atom in the 282 MHz ¹⁹F-NMR spectrum, whereas the same fluorine atom in the free amine is displaced to the low field at δ_{F}=−65.5 ppm.

The attempt was made to transfer 1 into the free amine by adding water. It was able to be shown that the addition of water to 1 in [D₆]-DMSO did not lead to a formation of the free amine at all. Free amines are the first products to arise from the hydrolysis of a metal amide, and would be detectable via the methods of analysis used. The pKₐ value of H-BPFPA amounts to 12.6 in DMSO at 25° C. In order to produce this comparably strong NH-acidic compound, a corresponding salt is usually treated with a strong acid such as aqueous HCl. Compound 1 is slightly hygroscopic, but no further hydrolysis is observed at room temperature.

### Practical Embodiment 10

### Hydrolysis of the Compounds (3) and (4)

As described in practical embodiment 9 for compound 1, the stability of compounds 3 and 4 against hydrolysis was shown with the aid of ¹⁹F-NMR spectroscopy by adding water to a [D₆]-DMSO solution of 3 and 4. While the signal for the para-fluorine atoms of the Pfp residue appears in the anions at δ_{F}=−168.2 ppm (3) and δ_{F}=−168.2 ppm (4), no traces of the corresponding sulfonamide acids in the NMR are found at δ_{F}=−163.9 ppm (H-PFTFSI) and δ_{F}=−165.8 ppm (H-PFNFSI)

### Thermogravimetric Analysis (TGA) and Differential Scanning Calorimetry (DSC)

Thermogravimetric analyses were carried out with a METTLER Toledo TGA/SDTA 851^{e} under a constant stream of nitrogen in aluminum crucibles.

Differential Scanning Calorimetry measurements were carried out with a METTLER Toledo DSC 821 under a constant stream of nitrogen in open aluminum crucibles.

### Practical Embodiment 11

### Thermogravimetric Analysis (TGA) and Differential Scanning Calorimetry (DSC) of the Compound (1)

The thermal stability of compound 1 was examined via thermogravimetric analysis (TGA) and Differential Scanning Calorimetry (DSC). The TGA shows the start of a loss of mass at 108° C. At 180°, a high loss of mass up to 15% of the original mass was observed (FIG. 4). In the heating phase, the DSC yields a glass transition at −31° C. and an endothermic phase transition at 87° C. with a transition enthalpy of 23.8 kJ/mol.

### Practical Embodiment 12

### Thermogravimetric Analysis (TGA) and Differential Scanning Calorimetry (DSC) of Compounds (3) and (4)

The thermal stability of compounds 3 and 4 was examined via thermogravimetric analysis (TGA) and Differential Scanning Calorimetry (DSC).

It was found that the insertion of a triflyl group (Tf, CF₃) or a nonaflyl group (Nf, C₄F₉) has a major influence on the thermal stability of the corresponding lithium salts.

Compound 3 begins to disintegrate at a temperature of 307° C. (0.05% weight loss), while the disintegration of compound 4 begins at 316° C. This thermal stability is sufficient for the desired electrochemical applications.

Finally, DSC measurements up to 10° C. were carried out under the disintegration point of both substances, wherein glass transitions alone took place in both cases. Compound 3 shows a glass transition at −8° C. while compound 4 shows the glass transition at 22° C.

The results of the TGA measurements are shown in FIG. 4.

### Practical Embodiment 13

### Impedance Spectroscopic Measurements

Impedance spectroscopic measurements were carried out on the lithium salts Li-PFTFSI and Li-PFNFSI in solid state in order to obtain information with regard to their lithium ion conductivity properties in solid state. For comparison purposes, impedance spectra of the known salt Li-TFSI were likewise recorded. The real part of the conductivity, σ′(v), was calculated from the frequency-dependent, complex impedance \hat{Z}(v)=Z′(v)−i·Z″(v) according to:

$${σ}^{′} ( v ) = \frac{d}{A} \cdot \frac{{Z}^{′} ( v )}{{[ {Z}^{′} ( v ) ]}^{2} + {[ {Z}^{″} ( v ) ]}^{2}} \qquad ( 1 )$$

d and A here characterize the thickness and surface of the sample, respectively.

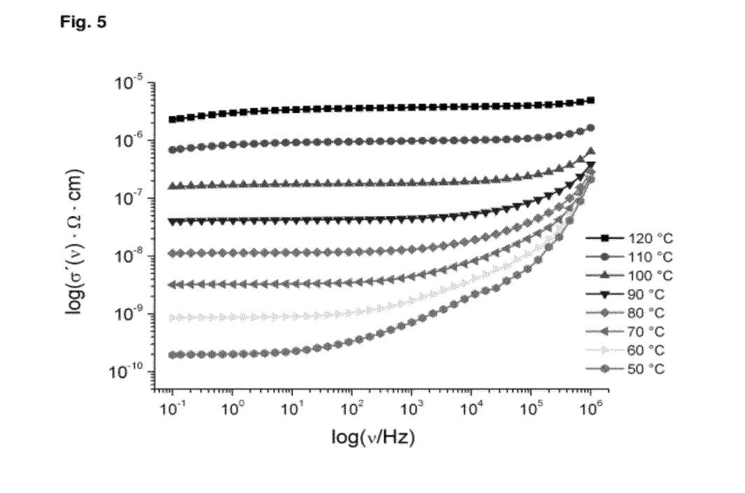

In FIG. 5 σ′(v) spectra for Li-PFTFSI at various temperatures are shown. The low frequency range of the spectrum is characterized by a plateau region between 50° C. and 100° C. In this area, σ′(v) is identical with the volume conductivity at direct current (bulk DC conductivity) σ_{dc}, which is reflected in the macroscopic transport of lithium ions. At higher frequencies, σ′(v) becomes dependent on frequency. This dispersive area contains additional information about localized movements of the lithium ions in the salts. At the two highest temperatures, the spectra under 10 Hz are characterized by a decrease of σ′(v) at a decreasing frequency. The plateau of the volume conductivity is therefore only detectable at frequencies above 10 Hz.

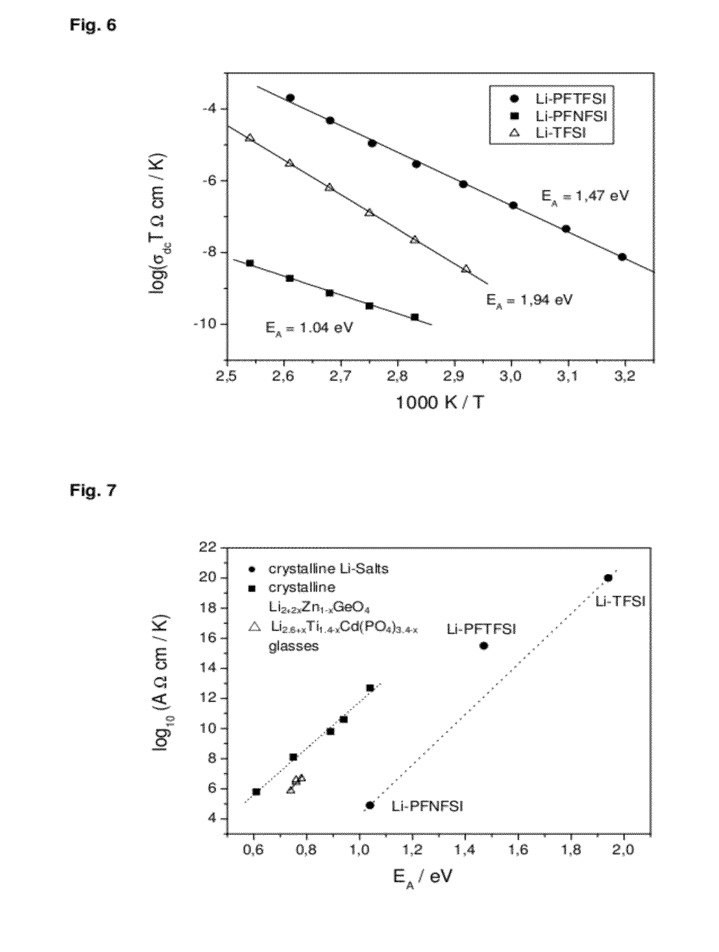

FIG. 6 shows an Arrhenius plot of the volume conductivity at direct current (bulk DC conductivity) σ_{dc} of all lithium salts. In the temperature range examined, the highest conductivity arose for Li-PFTFSI, the second highest for Li-TFSI and the lowest for Li-PFNFSI. The data were adjusted according to the Arrhenius law:

$${σ}_{dc} \cdot T = A \cdot exp ( \frac{{E}_{A}}{{k}_{B} T} ) \qquad ( 2 )$$

A and E_{A} here refer to the pre-exponential factor and the activation energy of the DC conductivity, respectively, wherein k_{B} is the Boltzmann constant.

The activation energy decreases in the sequence E_{A} (Li-TFSI)>E_{A}(Li-PFTFSI)>E_{A} (Li-PFNFSI). Remarkably, Li-PFNFSI, the salt with the lowest conductivity, also comprises the lowest activation energy.

In FIG. 7, the pre-exponential factor A is plotted logarithmically against the activation energy E_{A}. It is obvious that the pre-exponential factor rises with increasing activation energy. This property is in accordance with the known Meyer-Neldel rule (MN rule). This rule states that the following applies for a family of ionically conductive materials with similar structures:

log(A)=α·E_{A}+β (3)

Two known families of lithium ion conductors are contained in FIG. 7: polycrystalline LISICON materials of the composition Li₂₊₂ₓZn₁₋ₓGeO₄ and LISICON-type glasses of the composition Li_{2.6+x}Ti_{1.4-x}Cd(PO₄)_{3.4-x.} Both families are effectively described by the MN rule.

When considered from a theoretical standpoint, it is to be expected that materials follow the MN rule if the majority of the ions are trapped in low potentials, meaning that thermal activation is required in order to create mobile ions. An example is the formation of mobile interstitial ions and holes in crystals. In this case, the activation energy E_{A} can be written as the sum of two terms:

E_{A}=E_{A}^{act}+E_{A}^{mig} (4)

The first term E_{A}^{act} is the energy required for the thermal activation of the trapped ions, whereas the second term E_{A}^{mig} describes the activation energy for the migration of the activated ions (e.g. migration of defects in crystals). The activation of trapped ions is also characterized by an activation entropy S_{A}^{act}, which is taken into consideration in the pre-exponential factor A:

$$A = \frac{{N}_{v} \cdot {e}^{2} \cdot {a}^{2} \cdot {v}_{0}}{6 {k}_{B}} \cdot exp ( \frac{{S}_{A}^{act}}{{k}_{B}} ) = {A}_{0} \cdot exp ( \frac{{S}_{A}^{act}}{{k}_{B}} ) \qquad ( 5 )$$

Nᵥ and α here characterize the particle number density of all ions in the sample and the jump distance, respectively, while e is the elementary charge. The test frequency v₀ is suitable to be equalized with the oscillation frequency of the ions in their potential cages. The use of typical values for these variables leads to log(A₀·Ωcm/K)≈5.

The Meyer-Neldel rule is based on the assumption that

$${S}_{A}^{act} = \frac{{E}_{A}^{act}}{{T}_{0}} \qquad ( 6 )$$

This equation implies that at the temperature T₀, the free energy is zero for the activation of trapped ions. It follows from equations (4)-(6) that

$$log ( A ) = log ( {A}_{0} ) + \frac{{E}_{A} - {E}_{A}^{mig}}{{kT}_{0} \cdot ln ( 10 )} \qquad ( 7 )$$

In accordance with the MN rule (3), equation (7) is suitable to be used in order to define the parameters E_{A}^{mig} and T₀ from experimental data.

While the data from the compounds according to the present invention follow the the MN rule in a qualitative manner, the data do not comprise any linear relationship from log(A) against E_{A}. This implies that the similarities in the structure and in the mechanisms of ionic conduction are not sufficient to produce electrochemical behavior that follows the Meyer-Neldel rule exactly. Exact information regarding E_{A}^{mig} and T₀ is therefore unable to be obtained. However, equation (5) is suitable to be used to calculate the activation entropy S_{A}^{act}. Values from S_{A}^{act}=24·k_{B} for Li-PFTFSI and S_{A}^{act}=34,5·k_{B} for Li-TFSI were obtained. In the case of Li-PFNFSI, energy and entropy for the activation of lithium ions are negligible.

### FIGURE LEGENDS

FIG. 1

ORTEP plot of the compound 1, 30% probability of the ellipsoids.

Selected bond lengths [Å] and angle [°]:

Li1-N001 2.101(5), Li1-N101 2.084(5), Li2-N001 2.082(5), Li2-N101 2.110(5), F001-Li1 2.050(4), F105-Li1 2.224(4), F102″-Li1 2.032(5), F005″-Li1 2.677(5), F107-Li2 2.022(4), F010-Li2 2.152(4), F007′-Li2 2.043(4), F111′-Li2 2.605(5), N101-Li1-N001 102.9(2), N001-Li2-N101 102.7(2), Li1-N101-Li2 77.0(2), Li2-N001-Li1 77.3(2).

FIG. 2

ORTEP plot of the compound 2, 30% probability of the ellipsoids.

Selected bond lengths [Å] and angle [°]:

C1-C2 1.407(5), C1-N1 1.432(4), N1-C2 1.414(5), C7-N1 1.465(4), C2-N1-C1 115.6(3), C1-N1-C7 112.8(3), C2-N1-C7 113.7(3).

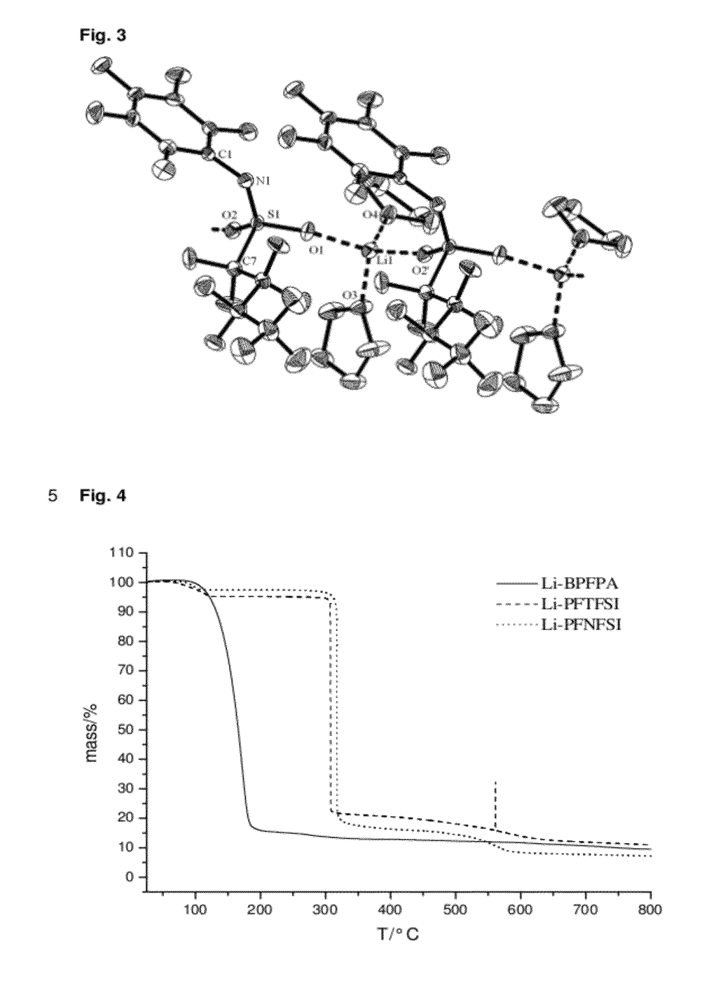

FIG. 3

ORTEP plot of the compound 4 (THF)₂, 30% probability of the ellipsoids.

Selected bond lengths [Å] and angle [°]:

Li1-O1 1.917(7), Li1-O2′ 1.957(7), Li1-O3 1.920(5), Li1-O4 1.926(8), S1-N1 1.521(3), S1-O1 1.438(3), S1-O2 1.450(3), O1-S1-O2 116.0(2), O1-S1-N1 111.3(2), O2-S1-N1 116.7(2).

FIG. 4

Thermogravimetric analysis of the compounds 1, 3 and 4.

Solid line: Li-BPFPA (1)

Dashed line: Li-PFTFSI (3)

Dotted line: Li-PFNFSI (4)

FIG. 5

Conductivity spectra of solid Li-PFTSI (3) at different temperatures.

FIG. 6

Arrhenius plot of the conductivity of different lithium salts.

FIG. 7

Meyer-Neldel plot of the pre-exponential factors and activation energies. For comparison purposes, data for crystalline and glass-like LISICON-type materials from the state of the art are shown.

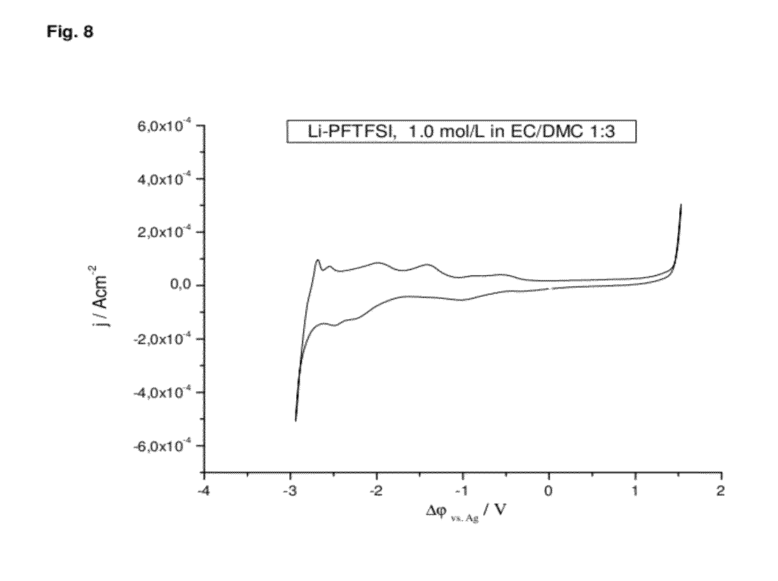

FIG. 8

Cyclic voltammogram of Li-PFTFSI, measured at a platinum working electrode against a silver reference electrode with a feed rate of 20 mV/s. The electrochemical window amounts to 4.2V.

EC/DMC refers to ethylene carbonate/dimethylcarbonate.

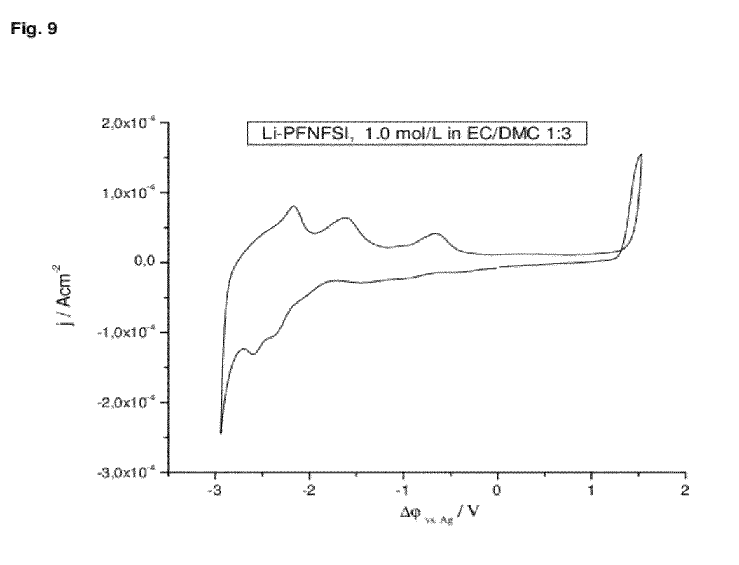

FIG. 9

Cyclic voltammogram of Li-PFNFSI, measured at a platinum working electrode against a silver reference electrode with a feed rate of 20 mV/s. The electrochemical window amounts to 4.2V.

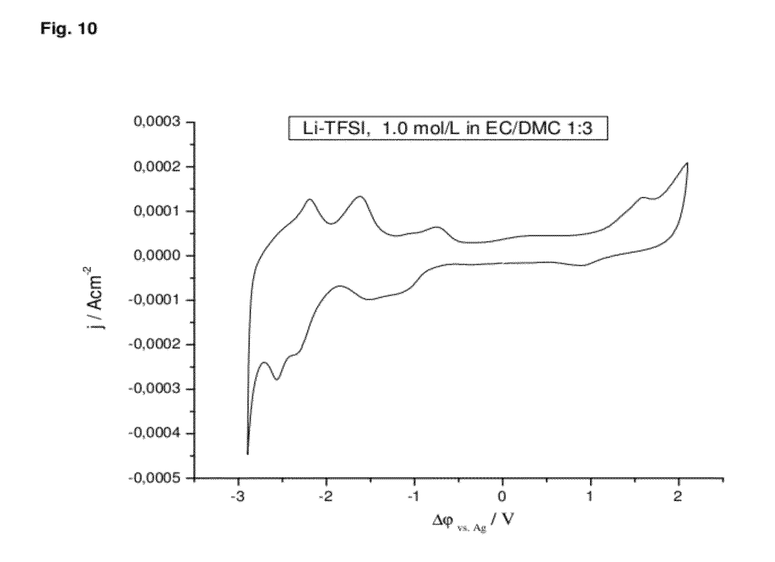

FIG. 10

Cyclic voltammogram of Li-TFSI, measured at a platinum working electrode against a silver reference electrode with a feed rate of 20 mV/s. The electrochemical window amounts to 4.9V.

1. A lithium salt comprising a lithium cation and a pentafluorophenylamide anion according to formula (I) 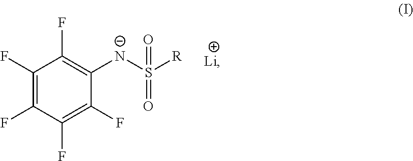
wherein R is selected from
linear or branched acyclic or cyclic alkyl groups with 1 to 20 carbon atoms which are not fluorinated, partially fluorinated or fully fluorinated,
aryl or benzyl groups with up to 20 carbon atoms, which are not fluorinated, partially fluorinated or completely fluorinated.

2. The lithium salt according to claim 1, wherein R is selected from the group consisting of fluorine, trifluoromethyl, and nonafluoro-n-butyl.

3. The lithium salt according to claim 1, wherein a solvent molecule is not coordinated to the lithium cation.

4. A method for the production of the lithium salt according to claim 1, the method comprising the steps:
a) dissolving the free acid of the pentafluorophenylamide in accordance with formula II 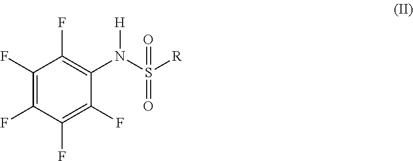
in a non-polar aprotic or dipolar aprotic solvent by heating,
b) mixing with an equimolar amount of lithium bis(trimethylsilyl)amide or a lithium organyl as a solid or in solution to obtain a mixture,
c) adjusting the obtained mixture of free acid consisting of the pentafluorophenylamide and the lithium trimethylsilylamide or the lithium organyl to a total concentration of these two reactants from 0.01 mol/L to 0.5 mol/L,
d) stirring the mixture at 80° C. for 12 hours,
e) cooling the mixture to room temperature,
f) removing volatile components from the mixture to provide a residue,
g) washing the residue with a non-polar aprotic or dipolar aprotic solvent and subsequent drying.

5. The method according to claim 4, wherein the lithium bis(trimethylsilyl)amide or the lithium organyl according to step b) is added as a solution.

6. The method according to claim 5, wherein the mixing in accordance with step b) comprises providing a solution of the lithium bis(trimethylsilyl)amide or the lithium organyl and subsequently adding the solution of the pentafluorophenylamide in accordance with step a).

7. The method according to claim 4, wherein the lithium bis(trimethylsilyl)amide or the lithium organyl according to step b) is added as a solid.

8. The method according to claim 5, wherein the residue is washed with a non-polar aprotic solvent.

9. The method according to claim 7, wherein the residue is washed with a dipolar aprotic solvent.
